(19) United States
(12) Patent Application Publication
(10) Pub. No.: US 2010/0274061 A1
(43) Pub. Date: Oct. 28, 2010

(54) OXYCHLORINATION CATALYST

(75) Inventors: Heiko Urtel, Frankenthal (DE); Henrik Junicke, Mannheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludswigshafen (DE)

(21) Appl. No.: 12/745,865

(22) PCT Filed: Dec. 2, 2008
(86) PCT No.: PCT/EP08/66609

§ 371 Date: Jun. 2, 2010

(30) Foreign Application Priority Data
Dec. 3, 2007 (EP) 07122109.7

(51) Int. Cl. C07C 17/013

(52) U.S. Cl. 570/243

(57) ABSTRACT

Process for the oxychlorination of ethylene to form 1,2-dichloroethane in the gas recycle mode with reduced accumulation of hydrogen, which comprises the following steps:
(A) introduction of ethylene, oxygen and hydrogen chloride as starting materials into an oxychlorination reactor, with the hydrogen chloride comprising hydrogen,
(B) reaction of the starting materials over a catalyst comprising
   from 3.5 to 12.5% by weight of copper as copper salt
   from 0.1 to 1.0% by weight of magnesium as magnesium salt
   from 0.1 to 2.0% by weight of potassium as potassium salt on a support material
      to form 1,2-dichloroethane and water with at least partial conversion of the hydrogen present into water,
(C) work-up of the reactor output and recirculation of the recycle gas to the oxychlorination reactor.

The present invention relates to a method of preventing the accumulation of hydrogen in oxychlorination reactions carried out in the gas recycle mode.

The oxychlorination of ethylene to form 1,2-dichloroethane (EDC) is a generally known process in which ethylene is reacted with hydrogen chloride and oxygen or with an oxygen-comprising gas (e.g. air) in the gas phase and usually in the presence of a catalyst. Here, use is frequently made of hydrogen chloride from the pyrolysis reaction of 1,2-dichloroethane (EDC), which generally comprises impurities (in particular acetylene). In the oxychlorination reaction, these lead to by-products (in particular polychlorinated compounds), as a result of which separation of the main product EDC from the oxychlorination reaction mixture is made more difficult. The hydrogen chloride is therefore subjected to a hydrogenation step in which the impurities are decomposed before being recirculated to the oxychlorination reaction. For this purpose, the hydrogen chloride is reacted with an up to five-fold excess of hydrogen, based on the impurities to be hydrogenated (in particular acetylene), in the presence of a hydrogenation catalyst. The hydrogen chloride used for the oxychlorination therefore also comprises hydrogen.

DE 1 618 701 describes an oxychlorination process in which the reaction gas is circulated.

In the oxychlorination process known as the “Mitsui-Toatsu process”, ethylene is reacted in the presence of oxygen and hydrogen chloride over a fluidized-bed catalyst comprising aluminum oxide and copper, with the molar ratios of the reaction gas being ethylene from 1.5 to 2.00:hydrogen chloride 2.00:oxygen from 0.5 to 1.00. The gas leaving the fluidized-bed reactor is separated by means of a condenser or another suitable apparatus into products and a gaseous mixture comprising unreacted starting materials and gaseous by-products such as carbon dioxide and carbon monoxide. Carbon dioxide and possibly residual hydrogen chloride are removed from the gas mixture by scrubbing with a basic solution. The resulting gas mixture, which comprises mainly ethylene, oxygen and carbon monoxide, is recirculated to the fluidized-bed reactor. Before it reenters the fluidized-bed reactor, ethylene, oxygen and hydrogen chloride are added to the recycle gas. High hydrogen chloride conversions are achieved in this process but the EDC selectivities achieved are generally unsatisfactory.

It is stated that the EDC selectivity of the oxychlorination can be improved by doping the copper-comprising catalyst with magnesium and/or potassium. Examples of such catalysts are described, for instance, in EP 1 464 395 and EP 1 666 145 for use in “single pass” reactors, with the catalysts used there having, in addition to the doping with magnesium and potassium, a specific concentration distribution of the catalyst metal over the catalyst support particle. For the purposes of the present invention, “single pass” processes are processes in which the reaction gas passes through the reactor only once and is not circulated in a recycle process.

However, to obtain an economical process, it is desirable for the process to be carried out in the recycle mode, so that unreacted starting materials are recirculated to the reactor.

The catalysts described in the prior art which have a high EDC selectivity are generally unsuitable for oxychlorination reactions in the gas recycle mode. Owing to the high selectivity, accumulation of hydrogen is observed since, despite its high reactivity in principle, hydrogen does not react to form subsequent products (generally water) and accumulates as a result of operation in the gas recycle mode. The high hydrogen content results in unsafe operating conditions and the risk of, for instance, explosions increases.

The hitherto customary oxychlorination processes operated in the gas recycle mode are therefore carried out using catalysts which comprise copper in concentrations of from 10 to 12% by weight, have a high activity and thus give a high hydrogen chloride conversion. Owing to the high activity, these catalysts catalyze not only the reaction of ethylene to form EDC but also the reaction (removal) of hydrogen to form water to a sufficient extent. However, the high activity of the catalyst is generally associated with unsatisfactory EDC selectivities; for instance, the formation of undesirable by-products such as carbon monoxide and carbon dioxide is also catalyzed.

It was thus an object of the invention to provide an oxychlorination process operated in the gas recycle mode, in which the accumulation of hydrogen in the reaction gas is prevented or at least greatly reduced. Unsafe operating conditions should be avoided. The EDC selectivity should, at a high hydrogen chloride conversion, be improved compared to oxychlorination processes which are known from the prior art and are likewise operated in the recycle mode. The selectivity should be comparable with selectivities known for catalysts from the prior art. The process should be very simple and economical and should preferably use a catalyst which is preferably simple to produce and inexpensive.

The object is achieved by a process for the oxychlorination of ethylene to form 1,2-dichloroethane in the gas recycle mode with reduced accumulation of hydrogen, which comprises the following steps:
(A) introduction of ethylene, oxygen and hydrogen chloride as starting materials into an oxychlorination reactor, with the hydrogen chloride comprising hydrogen,
(B) reaction of the starting materials over a catalyst comprising
   from 3.5 to 12.5% by weight of copper as copper salt
   from 0.1 to 1.0% by weight of magnesium as magnesium salt
   from 0.1 to 2.0% by weight of potassium as potassium salt on a support material
      to form 1,2-dichloroethane and water with at least partial conversion of the hydrogen present into water,
(C) work-up of the reactor output and recirculation of the recycle gas to the oxychlorination reactor.

For the purposes of the present invention, all percentages by weight are based on the total weight of the catalyst including the support material.

For the purposes of the present invention, the reaction gas is always the sum of fresh gas and recycle gas; recycle gas is any gas mixture which is obtained after passage through the reactor and, if appropriate, after work-up steps and is recirculated to the reactor.

The hydrogen chloride conversion is a measure of the activity of the catalyst used in the process and is defined as:

hydrogen chloride conversion [%]={hydrogen chloride reacted/fresh hydrogen chloride fed in}·100

the EDC selectivity is defined as:

EDC selectivity [%]={EDC formed/ethylene reacted}·100

To prevent accumulation of hydrogen in the reaction gas, it is necessary, in a single pass through the reactor, to remove an amount of hydrogen which is at least as great as the amount of fresh hydrogen fed in.

For the purposes of the present invention, reduced accumulation of hydrogen means that the accumulation of hydrogen is either prevented completely, i.e. hydrogen is completely removed in a single pass through the reactor and no hydrogen is present in the recycle gas, or that the hydrogen is removed in a single pass through the reactor to such an extent that the hydrogen content of the recycle gas stabilizes at values which make safe operating conditions possible and lead to a hydrogen content in the reaction gas of not more than 2.0%, preferably not more than 1.8% and particularly preferably 1.5%.

It has surprisingly been found that the use of catalysts comprising copper, magnesium and potassium in ratios within the range according to the invention enable high product selectivities to be achieved while at the same time catalyzing the reaction of hydrogen to form water to a sufficient extent. Accumulation of hydrogen is thus prevented in gas recycle operation, so that unsafe operating conditions are avoided. The use of these catalysts suppresses the formation of undesirable by-products and good EDC selectivities are achieved at a high hydrogen chloride conversion.

The process of the invention thus combines prevention of the accumulation of hydrogen in the reaction gas with good EDC selectivity and at the same time a high hydrogen chloride conversion. In addition, the process of the invention increases process safety.

The catalyst used in the process of the invention comprises metal salts in such proportions on a support material that it comprises from 3.5 to 12.5% by weight of copper as copper salt, from 0.1 to 1.0% by weight of magnesium as magnesium salt and from 0.1 to 2.0% by weight of potassium as potassium salt, where all percentages by weight are based on the total weight of the catalyst including the support material.

In a preferred embodiment, the catalyst used in the process of the invention comprises metal salts in such proportions on a support material that it comprises from 3.5 to 12.5% by weight of copper as copper salt, from 0.1 to 0.9% by weight of magnesium as magnesium salt and from 0.1 to 2.0% by weight of potassium as potassium salt, where all percentages by weight are based on the total weight of the catalyst including the support material.

In another preferred embodiment, the catalyst used in the process of the invention comprises metal salts in such proportions on a support material that it comprises from 3.5 to 12.5% by weight of copper as copper salt, from 0.2 to 0.8% by weight of magnesium as magnesium salt and from 0.1 to 2.0% by weight of potassium as potassium salt, where all percentages by weight are based on the total weight of the catalyst including the support material.

In a more preferred embodiment, the catalyst used in the process of the invention comprises metal salts in such proportions on a support material that it comprises from 3.5 to 12.5% by weight of copper as copper salt, from 0.3 to 0.7% by weight of magnesium as magnesium salt and from 0.1 to 2.0% by weight of potassium as potassium salt, where all percentages by weight are based on the total weight of the catalyst including the support material.

In another more preferred embodiment, the catalyst used in the process of the invention comprises metal salts in such proportions on a support material that it comprises from 3.5 to 12.5% by weight of copper as copper salt, from 0.3 to 0.7% by weight of magnesium as magnesium salt and from 0.6 to 0.9% by weight of potassium as potassium salt, where all percentages by weight are based on the total weight of the catalyst including the support material.

In a particularly preferred embodiment, the catalyst used in the process of the invention comprises metal salts in such proportions on a support material that it comprises from 7.5 to 9.0% by weight of copper as copper salt, from 0.3 to 0.7% by weight of magnesium as magnesium salt and from 0.6 to 0.9% by weight of potassium as potassium salt, where all percentages by weight are based on the total weight of the catalyst including the support material.

The catalyst used in the process of the invention may, if appropriate, comprise further metals in the form of metal salts or in elemental form, for example additional alkali metals, additional alkaline earth metals, metals of the rare earths, transition metals and noble metals such as gold, ruthenium, platinum and palladium.

The specific surface area (BET) of the catalyst used in the process of the invention is preferably in the range from 60 to 200 m²/g, more preferably from 80 to 145 m²/g, in particular in the range from 90 to 130 m²/g (the BET surface area is determined in accordance with DIN 66131). The pore volume of the fluidized-bed catalyst is in the range from 0.2 to 0.5 cm³/g (the pore volume is determined in accordance with DIN 66133). The tamped density of the catalyst according to the invention is in the range from 900 to 1200 g/l, preferably in the range from 950 to 1150 g/l. Particular preference is given to catalysts having a tamped density greater than 1000 g/l. The average particle size (D50) of the catalyst is from 45 to 75 μm.

The metal concentrations, based on the total weight of the catalyst including support material, are set by known methods with which those skilled in the art are familiar, by impregnation or coprecipitation. The metal concentration is preferably set by impregnation.

To carry out the impregnation, the required amounts of the metal salts, preferably in the form of chlorides, nitrates, acetates, hydroxides or carbonates, are dissolved in water. Particular preference is given to using metal chlorides. The aqueous solution is applied to the support material. The support material is, if appropriate, filtered off and dried. The amount of water is preferably selected so that it corresponds to from about 70 to 90% of the water uptake of the support material. In this case, filtration is not necessary.

Drying is carried out at room temperature or at temperatures in the range from 100 to 200° C. in the presence of air or protective gas. Preference is given to carrying out drying in the presence of nitrogen at temperatures in the range from 110 to 180° C.

The process of the invention for preparing 1,2-dichloroethane can be carried out using the known techniques and reaction conditions which are generally established in the prior art. For this purpose, ethylene, hydrogen chloride and oxygen are brought into contact in the gas phase with the catalyst according to the invention.

All oxychlorination reactors in which the reaction gas is circulated are suitable for the process of the invention. Suitable reactors are tube reactors, fixed-bed reactors and fluidized-bed reactors. Fluidized-bed reactors are preferred.

After the process of the invention, the composition of the reaction gas is in the region of the following molar ratios: ethylene from 1.0 to 2.0:hydrogen chloride 2.0:oxygen from 0.5 to 1.0.

In a preferred embodiment of the process of the invention, the composition of the reaction gas is in the region of the following molar ratios: ethylene from 1.55 to 2.0:hydrogen chloride 2.0:oxygen from 0.5 to 0.75.

Suitable oxygen-comprising gases are air or oxygen, with preference being given to oxygen. Hydrogen chloride from the customary sources can in principle be used as hydrogen chloride. For practical reasons, preference is given to hydrogen chloride which is obtained from EDC pyrolysis and comprises hydrogen concentrations of from 0 to 2% by volume.

The temperature in the reactor is generally from 80 to 300° C., preferably from 200 to 280° C., in particular from 210 to 260° C.

The pressure in the reactor is from 1 to 20 bar, preferably from 1 to 8 bar, in particular from 1 to 5 bar.

Suitable support materials for the catalyst used in the process of the invention are aluminum oxides such as alpha-aluminum oxide, beta-aluminum oxide and gamma-aluminum oxide, activated aluminum oxides, silicon oxides such as silicates, silica gels, silicic acid and kieselguhr, graphite, metal oxides, zirconium oxides, zeolites, water glass, pumice, clays, aluminas and mixtures of the abovementioned materials. Preference is given to using gamma-aluminum oxide.

The specific surface area of the support material before deposition of metal salt is preferably in the range from 20 to 400 m²/g, more preferably from 75 to 200 m²/g (the BET surface area is determined in accordance with DIN 66131). Customary support materials preferably have pore volumes in the range from 0.15 to 0.75 cm³/g (the determination of the pore volume is carried out in accordance with DIN 66133). The tamped density of the support material is in the range from 600 to 950 g/l, preferably in the range from 700 to 850 g/l (the tamped density is a measure of the density of particulate solids. The tamped density is determined on an STAV 2003 tamping volumeter from JEL. The sample material is introduced during the first 350 shaking cycles. The tamped volume is determined after a further 350 cycles). The average particle size (D50) of the support material is from 45 to 75 μm (the particle sizes are determined by means of a Mastersizer S analytical instrument from Malvern; measurement parameters for the instrument: gas velocity 80 m/s, scattering model 3 $$ A (Fraunhofer), focal length 300 mm, beam length 10 mm, dispersion pressure 0.5 bar; Iso 13320).

In a particular embodiment of the process of the invention, the oxychlorination is carried out over a fluidized-bed catalyst in a fluidized-bed reactor, with the molar ratios of the reaction gas being ethylene from 1.55 to 2.00:hydrogen chloride 2.00:oxygen from 0.5 to 0.75. The gas leaving the fluidized-bed reactor is separated by means of a condenser or another suitable apparatus into products and a gaseous mixture comprising unreacted starting materials and gaseous by-products such as carbon dioxide and carbon monoxide. Carbon dioxide and possibly residual hydrogen chloride are removed from the gas mixture by scrubbing with a basic solution. The gas mixture obtained in this way, which comprises mainly ethylene, oxygen and carbon monoxide, is recirculated to the fluidized-bed reactor. Before it reenters the fluidized-bed reactor, ethylene, oxygen and hydrogen chloride are added to the recycle gas in such amounts that the reaction gas has the above-described molar ratios.

The invention is illustrated by the following examples without being restricted thereto.

### EXAMPLES

The catalysts used in the process of the invention were obtained by impregnation of the support material with an aqueous solution, as described below:

The amounts of the appropriate metal chlorides necessary for setting the desired metal ratios were dissolved in a small amount of water. Further water was added to make up the solution to a total volume of about 90% of the maximum water uptake of the support material used. This solution was sprayed onto a gamma-aluminum oxide support having a BET surface area of 170 m²/g in an impregnation drum. The average particle sizes of the supports (d50) were in the range from 45 to 75 p.m. After the addition was complete, the impregnated supports were dried at 110-180° C. in the presence of nitrogen for 16 hours.

To carry out comparative experiments, the catalysts 1 to 3 according to the invention and the comparative catalysts 4 to 8 were produced in the above-described manner. The compositions of the catalysts are summarized in table 1.

TABLE 1

| | Examples | | | Comparative examples | | | | |
|---|---|---|---|---|---|---|---|---|
| Catalyst | 1 | 2 | 3 | 4 | 5 | 6 | 7 | 8 |
| Cu [% by wt.] | 6.0 | 8.1 | 8.2 | 8.0 | 10.5 | 5.8 | 9.9 | 12 |
| Mg [% by wt.] | 0.1 | 0.9 | 0.49 | 1.5 | 1.75 | 1.4 | — | — |
| K [% by wt.] | 0.8 | 0.7 | 0.83 | 0.8 | 0.85 | 0.81 | 0.93 | — |
| Au [% by wt.] | 0.0009 | 0.0009 | — | — | — | 0.010 | — | — |
| BET [m²/g] | 112 | 110 | 115 | 112 | 92 | 105 | 141 | 260 |

The reactors are oil-heated glass reactors having an internal reactor diameter of 3 or 4 cm.

The reaction temperature was measured and controlled by means of a temperature sensor above the hot spot of the reactor. All experiments were carried out using fixed settings of the gas flows at the temperatures set.

A reactor which had an internal diameter of 4 cm and was charged with 240 g of catalyst was used for determining the hydrogen conversion. The reaction was carried out at a pressure of 2.4 bar and a hydrogen chloride throughput of 425 standard I/kg of cat/h. As reaction gas, use was made of a mixture having the following composition: ethylene 23.2% by volume, hydrogen 0.95% by volume, oxygen 9.1% by volume, hydrogen chloride 28.1% by volume, balance nitrogen. This corresponds to a stoichiometry of ethylene:hydrogen chloride:oxygen of 1.65:2:0.65 (t 0.02). The results are summarized in table 2.

TABLE 2

| | Catalyst | | | |
|---|---|---|---|---|
| | 3 | 6 | 7 | 8 |
| Cu [% by wt.] | 8.2 | 5.8 | 9.9 | 12.0 |
| Mg [% by wt.] | 0.49 | 1.40 | — | — |
| K [% by wt.] | 0.83 | 0.81 | 0.93 | — |
| Au [% by wt.] | — | 0.010 | — | — |
| BET [m²/g] | 115 | 105 | 141 | 260 |
| Conversion of HCl, 220° C. [%] | 98.7 | 70.2 | 99.7 | 97.4 |
| HCl conc. in the offgas [% by vol.] | 0.5 | 11.7 | 0.1 | 1.1 |
| H₂ conversion, 220° C. [%] | 9.0 | 2.2 | 20.0 | 14.5 |
| Conversion of HCl, 230° C. [%] | 99.2 | 73.7 | 99.4 | 95.5 |
| HCl conc. in the offgas [% by vol.] | 0.3 | 10.4 | 0.2 | 1.8 |
| H₂ conversion, 230° C. [%] | 9.9 | 1.2 | 27.7 | 13.8 |
| Conversion of HCl, 245° C. [%] | 99.5 | 87.9 | 98.6 | 87.0 |
| HCl conc. in the offgas [% by vol.] | 0.2 | 5.0 | 0.5 | 5.0 |
| H₂ conversion, 245° C. [%] | 21.7 | 8.3 | 32.5 | 14.8 |

The experiments show that the catalyst 3 according to the invention achieves significantly higher degrees of removal of hydrogen compared to catalyst 6. The degrees of removal of hydrogen of catalyst 3 are lower than those of catalysts 7 and 8, but sufficiently high to prevent accumulation of hydrogen in the recycle gas.

A reactor which had an internal diameter of 4 cm and was charged with 240 g of catalyst was used for determining the EDC selectivity. The reaction was carried out at a pressure of 2.4 bar and a hydrogen chloride throughput of 500 standard I/kg of cat/h. As reaction gas, use was made of a mixture having the following composition: ethylene 24.6% by volume, oxygen 9.7% by volume, hydrogen chloride 29.9% by volume, balance nitrogen. This corresponds to a stoichiometry of ethylene:hydrogen chloride:oxygen of 1.65:2:0.65 (±0.02). The results are summarized in table 3.

TABLE 3

| | Catalyst | | | |
|---|---|---|---|---|
| | 3 | 6 | 7 | 8 |
| Cu [% by wt.] | 8.2 | 5.8 | 9.90 | 12.00 |
| Mg [% by wt.] | 0.49 | 1.40 | — | — |
| K [% by wt.] | 0.83 | 0.81 | 0.93 | — |
| Au [% by wt.] | — | 0.010 | — | — |
| BET [m²/g] | 115 | 105 | 141 | 260 |
| 215° C. | | | | |
| Conversion of HCl [%] | 99.0 | 66.4 | | 99.7 |
| HCl conc. in the offgas [% by vol.] | 0.3 | 10.5 | | 0.1 |
| EDC selectivity [%] | 99.58 | 99.54 | | 94.58 |
| Yield (CO + CO₂) [%] | 0.1 | <0.1 | | 2.3 |
| 230° C. | | | | |
| Conversion of HCl [%] | 97.0 | 78.6 | | 99.0 |
| HCl conc. in the offgas [% by vol.] | 0.9 | 6.4 | | 0.3 |
| EDC selectivity [%] | 99.49 | 99.56 | | 92.5 |
| S (CO + CO₂) [%] | 0.1 | <0.1 | | 3.8 |
| 235° C. | | | | |
| Conversion of HCl [%] | 97.6 | 80.7 | 98.2 | 98.3 |
| HCl conc. in the offgas [% by vol.] | 0.2 | 5.8 | 0.6 | 0.5 |
| EDC selectivity [%] | 98.3 | 99.12 | 95.1 | 87.1 |
| S (CO + CO₂) [%] | 0.39 | <0.1 | 1.0 | 3.84 |
| 240° C. | | | | |
| Conversion of HCl [%] | 96.3 | 83.6 | | 96.7 |
| HCl conc. in the offgas [% by vol.] | 1.1 | 4.9 | | 1.0 |
| EDC selectivity [%] | 99.14 | 99.56 | | 89.7 |
| S (CO + CO₂) [%] | 0.31 | <0.1 | | 4.5 |
| 255° C. | | | | |
| Conversion of HCl [%] | 95.0 | 87.6 | | 89.0 |
| HCl conc. in the offgas [% by vol.] | 1.5 | 3.7 | | 3.3 |
| EDC-selectivity [%] | 98.28 | 98.48 | | 80.7 |
| S (CO + CO₂) [%] | 0.75 | 0.3 | | 4.6 |

The experiments show that the catalyst 3 according to the invention achieves significantly higher EDC selectivities compared to catalysts 7 and 8 at a high hydrogen chloride conversion and removes hydrogen to a sufficient extent over a wide temperature range.

1-13. (canceled)

14. A process for the oxychlorination of ethylene to form 1,2-dichloroethane in the gas recycle mode with reduced accumulation of hydrogen, which comprises the following steps:
(A) introduction of ethylene, oxygen and hydrogen chloride as starting materials into an oxychlorination reactor, with the hydrogen chloride comprising hydrogen,
(B) reaction of the starting materials over a catalyst comprising
from 3.5 to 12.5% by weight of copper as copper salt
from 0.1 to 1.0% by weight of magnesium as magnesium salt
from 0.1 to 2.0% by weight of potassium as potassium salt on a support material
to form 1, 2-dichloroethane and water with at least partial conversion of the hydrogen present into water,
(C) work-up of the reactor output and recirculation of the recycle gas to the oxychlorination reactor.

15. The process according to claim 14, wherein the catalyst comprises from 7.5 to 9.0% by weight of copper as copper salt, from 0.3 to 0.7% by weight of magnesium as magnesium salt and from 0.6 to 0.9% by weight of potassium as potassium salt on a support material.

16. The process according to claim 14, wherein the support material of the catalyst is selected from the group consisting of aluminum oxides, alpha-aluminum oxide, beta-aluminum oxide, gamma-aluminum oxide, activated aluminum oxides, silicon oxides such as silicates, silica gels, silicic acid and kieselguhr, graphite, metal oxides, zirconium oxides, zeolites, water glass, pumice, clays, aluminas and mixtures of the abovementioned materials.

17. The process according to claim 14, wherein the catalyst comprises gamma-aluminum oxide as support material.

18. The process according to claim 15, wherein the catalyst comprises gamma-aluminum oxide as support material.

19. The process according to claim 14, wherein the catalyst has a BET surface area in the range from 60 to 200 m2/g.

20. The process according to claim 15, wherein the catalyst has a BET surface area in the range from 60 to 200 m2/g.

21. The process according to claim 14, wherein the catalyst has a BET surface area in the range from 80 to 145 m2/g.

22. The process according to claim 14, wherein the catalyst has a BET surface area in the range from 100 to 130 m2/g.

23. The process according to claim 14, wherein the catalyst has a tamped density of greater than 1000 g/l.

24. The process according to claim 14, wherein the catalyst has an average particle size (D50) in the range from 45 to 75 μm.

25. The process according to claim 15, wherein the catalyst has an average particle size (D50) in the range from 45 to 75 μm.

26. The process according to claim 14, wherein the starting materials are introduced into the reactor in the molar ratios ethylene from 1.00 to 2.0:hydrogen chloride 2.0:oxygen from 0.5 to 1.0.

27. The process according to claim 15, wherein the starting materials are introduced into the reactor in the molar ratios ethylene from 1.00 to 2.0:hydrogen chloride 2.0:oxygen from 0.5 to 1.0.

28. The process according to claim 14, wherein the starting materials are introduced into the reactor in the molar ratios ethylene from 1.55 to 2.0:hydrogen chloride 2.0:oxygen from 0.5 to 0.75.

29. The process according to claim 15, wherein the starting materials are introduced into the reactor in the molar ratios ethylene from 1.55 to 2.0:hydrogen chloride 2.0:oxygen from 0.5 to 0.75.

30. The process according to claim 14, wherein the oxychlorination reactor is a fluidized-bed reactor.

31. The process according to claim 15, wherein the oxychlorination reactor is a fluidized-bed reactor.

32. A process for prevent accumulation of hydrogen in a process for the oxychlorination of ethylene to form 1,2-dichloroethane in the gas recycle mode as described in claim 14, comprising utilizing a catalyst comprising
from 3.5 to 12.5% by weight of copper as copper salt
from 0.1 to 1.0% by weight of magnesium as magnesium salt
from 0.1 to 2.0% by weight of potassium as potassium salt on a support material.

33. A process for preventing accumulation of hydrogen in a process for the oxychlorination of ethylene to form 1,2-dichloroethane in the gas recycle mode as described in claim 15, comprising utilizing a catalyst comprising
from 3.5 to 12.5% by weight of copper as copper salt
from 0.1 to 1.0% by weight of magnesium as magnesium salt
from 0.1 to 2.0% by weight of potassium as potassium salt on a support material.
